(19) United States
(12) Patent Application Publication
(10) Pub. No.: US 2008/0031959 A1
(43) Pub. Date: Feb. 7, 2008

(54) ANTI-MIGRAINE ORAL SPRAY FORMULATIONS AND METHODS

(76) Inventors: Frank E. Blondino, Easton, PA (US); Carrie Chen, East Brunswick, NJ (US); Howard Malitz, Flemington, NJ (US); Foyeke Opawale, Flemington, NJ (US)

Correspondence Address: DICKSTEIN SHAPIRO LLP, 1825 EYE STREET NW, Washington, DC, 20006-5403, US

(21) Appl. No.: 11/829,396

(22) Filed: Jul. 27, 2007

Related U.S. Application Data
(60) Provisional application No. 60/833,847, filed on Jul. 28, 2006.

(51) Int. Cl. A61K 31/405; A61K 9/14; A61P 25/06

(52) U.S. Cl. 424/489; 514/415

(57) ABSTRACT

Formulations of an active pharmaceutical agent suitable for oral spray administration for absorption through the oral mucosa and related methods of preparation and administration are provided. Preferred embodiments provide sumatriptan succinate in a potassium phosphate buffer, wherein when a unit dose volume of about 50 to about 600 mcL of the oral spray composition is sprayed, a blood concentration of greater than about 5 ng/ml of sumatriptan is reached within about six minutes post dosing.

This application claims priority to U.S. Provisional Patent Application No. 60/833,847, filed on Jul. 28, 2006, the disclosure of which is incorporated by reference herein in its entirety.

### FIELD OF THE INVENTION

The field of the this invention is anti-migraine oral spray pharmaceutical formulations, methods of manufacturing such formulations, and their use for obtaining fast blood levels and pharmaceutical effects of the active ingredient via absorption to the systemic circulatory system through the oral mucosa in human and non-human mammals.

### BACKGROUND OF THE INVENTION

There are several limitations associated with administration of pharmaceutically active compounds through the gastrointestinal (GI) tract. The harsh environment of the gastrointestinal tract may alter the pharmaceutically active ingredient and decrease the available dosage. Metabolism by the liver may also limit the available dosage. Furthermore, patients with gastrointestinal sensitivities may have undesired side effects resulting from the gastrointestinal route of delivery.

Oral sprays may provide substantial benefits compared to GI and other modes of drug administration. These benefits depend on the particular dose, formulation and active ingredient, among other variables, and may include faster appearance of the pharmaceutically active ingredient in the blood, improved dosage reliability, improved safety profile, and increased bioavailability.

What is needed are stable oral spray formulations in suitable unit dose volumes for improving the speed of onset of pharmaceutical activity via absorption to the systemic circulatory system through the oral mucosa.

### SUMMARY OF THE INVENTION

The present invention provides stable spray formulations of an active pharmaceutical agent which may be provided in a volume effective for administration to the systemic circulatory system through the oral mucosa. Preferred embodiments of the invention provide oral spray compositions comprising a selective 5-hydroxytryptamine receptor subtype agonist, a buffer, and water, wherein when a unit dose volume of about 100 to 300 mcL of the pharmaceutical composition is sprayed, the blood concentration of the pharmaceutical composition is at least about 5 ng/ml at or before about six minutes post-dosing. In a preferred embodiment, the unit dose volume is about 50 to 600 mcL, most preferably about 240 mcL.

Particularly preferred embodiments of the invention provide formulations comprising sumatriptan and pharmaceutically acceptable salts thereof suitable for oral administration, and related methods of preparation and administration of sumatriptan formulations. In one embodiment, sumatriptan is formulated in a concentration of about 5 to 20% w/w, more preferably 7 to 15%, and most preferably about 11% w/w of the succinate salt. A particularly preferred sumatriptan formulation is propellant free and comprises sumatriptan succinate, monobasic potassium phosphate, sodium benzoate, sodium hydroxide, sucralose, acesulfame potassium, and purified water. Optional flavoring or taste-masking agents may be added.

Further embodiments of the invention provide methods for attaining fast onset of a pharmaceutically effective amount of a selective 5-hydroxytryptamine receptor subtype agonist by delivery through the oral mucosa of a mammal in need of treatment, wherein a peak blood level concentration of greater than about 5 ng/ml of the selective 5-hydroxytryptamine receptor subtype agonist is achieved within six minutes post dosing. In another embodiment, a peak blood level concentration of greater than about 10 ng/ml is achieved within six minutes post dosing. In yet another embodiment, an area under the curve (AUC) of greater than about 0.4 [(ng/ml)*h] is achieved after about six minutes post dosing with a selective 5-hydroxytryptamine receptor subtype agonist.

Other embodiments of the invention provide methods of delivering a pharmaceutically effective amount of a sumatriptan-containing formulation to the systemic circulatory system of a mammal via actuation of a spray pump adapted for administration of the formulation to the oral mucosal surfaces. In yet another embodiment, the spray pump delivers the equivalent of about 10 mg of sumatriptan base per 120 mcL actuation. In one embodiment, the dose is delivered by more than one actuation of the spray pump.

Additional embodiments of the invention provide methods for treating migraine by administering to an animal or human subject in need thereof an oral spray composition according to the invention. Preferred embodiments administer a spray volume of about 50-600 mcL, preferably about 120-360 mcL, and more preferably about 240 mcL to the oral mucosa. In another embodiment, the volume of spray contains a dose of sumatriptan in the range from about 5 to about 40 mg per dose, preferably about 10 to about 30 mg, and more preferably about 20 mg.

Additional features and advantages of the invention will be set forth in the description which follows and will be apparent from the description or may be learned by practice of the invention.

### BRIEF DESCRIPTION OF THE DRAWINGS

FIG. 1 shows the percent total impurities versus time for taste masked 11.23% w/w sumatriptan succinate formulation (Formulation B) under three stability conditions (25° C./60 RH, 30° C./65 RH, and 40° C./75 RH);

FIG. 2 shows the blood concentration of sumatriptan in ng/ml from 0 to 480 minutes post-dosing with a 20 mg lingual formulation, a 30 mg lingual formulation, or a 50 mg tablet;

FIG. 3 shows the blood concentration of sumatriptan in ng/ml from 0 to 15 minutes post-dosing with a 20 mg lingual formulation, a 30 mg lingual formulation, or a 50 mg tablet;

FIG. 4 shows the AUC (area under the curve) for sumatriptan in [(ng/ml)*h] from 0 to 15 minutes post-dosing with a 20 mg lingual formulation, a 30 mg lingual formulation, or a 50 mg tablet;

FIG. 5 shows the percentage of subjects with at least a 2-Point Reduction in headache severity rating from 30 to 120 minutes after receiving a 50 mg sumatriptan tablet, a 100 mg sumatriptan tablet, a 20 mg sumatriptan lingual spray, a 30 mg sumatriptan lingual spray, or a 40 mg sumatriptan lingual spray;

FIG. 6 shows the percentage of subjects with complete pain relief from 30 to 120 minutes after receiving a 50 mg sumatriptan tablet, a 100 mg sumatriptan tablet, a 20 mg sumatriptan lingual spray, a 30 mg sumatriptan lingual spray, or a 40 mg sumatriptan lingual spray;

FIG. 7 shows the percentage of subjects with no disability from 30 to 120 minutes after receiving a 50 mg sumatriptan tablet, a 100 mg sumatriptan tablet, a 20 mg sumatriptan lingual spray, a 30 mg sumatriptan lingual spray, or a 40 mg sumatriptan lingual spray;

FIG. 8 shows the rate of response to treatment after receiving a 50 mg sumatriptan tablet, a 100 mg sumatriptan tablet, a 20 mg sumatriptan lingual spray, a 30 mg sumatriptan lingual spray, or a 40 mg sumatriptan lingual spray from 0 to 240 minutes post-dosing;

FIG. 9 shows the rate of complete pain relief achieved in subjects after receiving a 50 mg sumatriptan tablet, a 100 mg sumatriptan tablet, a 20 mg sumatriptan lingual spray, a 30 mg sumatriptan lingual spray, or a 40 mg sumatriptan lingual spray from 0 to 240 minutes post-dosing;

FIG. 10 shows the rate of disability-free relief achieved by subjects after receiving a 50 mg sumatriptan tablet, a 100 mg sumatriptan tablet, a 20 mg sumatriptan lingual spray, a 30 mg sumatriptan lingual spray, or a 40 mg sumatriptan lingual spray from 0 to 240 minutes post-dosing; and

FIG. 11 shows the Cmax for the indicated dosage and route of administration in ng/ml.

### DETAILED DESCRIPTION OF THE INVENTION

Reference will now be made in detail to the presently preferred embodiments of the invention, which, together with the following examples, serve to explain the principles of the invention. It is to be understood that the application of the teachings of the present invention to a specific problem or environment will be within the capabilities of one having ordinary skill in the art in light of the teachings contained herein. Illustrative embodiments of the products of the present invention and processes for their preparation and use appear in the following examples.

Preferred embodiments of the present invention provide stable, aqueous, pharmaceutical compositions comprising a therapeutically effective amount of a selective 5-hydroxytryptamine receptor subtype agonist. In a preferred embodiment, the selective 5-hydroxytryptamine receptor subtype agonist is sumatriptan. A preferred concentration of sumatriptan succinate in the product is about 5 to 15% w/w, most preferably between about 10 and 14% w/w. Other selective 5-hydroxytryptamine receptor subtype agonists suitable for use in accordance with the invention include, for example, almotriptan, eletriptan, frovatriptan, naratriptan, rizatriptan and zolmitriptan. A preferred concentration of sumatriptan in other selective 5-hydroxytryptamine receptor subtype agonists suitable for use in accordance with the invention (e.g., almotriptan, eletriptan, frovatriptan, naratriptan, rizatriptan and zolmitriptan) is about 1 to 15%.

Sumatriptan as a free base, succinate salt, or other salt form is indicated for the acute treatment of migraine headaches with or without aura in adults. Sumatriptan is a selective 5-hydroxytryptamine receptor subtype agonist chemically designated as 3-[2-(dimethylamino)ethyl]-N-methyl-1H-indole-5-methanesulfonamide, the succinate salt having the following structure:

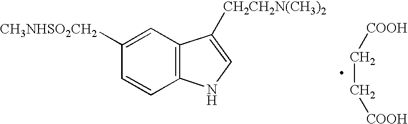

Sumatriptan activates the vascular 5-HT1 receptor subtype present on cranial arteries in both dog and primate, on the human basilar artery, and in the vasculature of human dura matter. The vascular 5-HT1 receptor subtype also mediates vasoconstriction. It is believed that this action in humans correlates with the relief of migraine headache. From animal studies it has been determined that sumatriptan also activates 5-HT1 receptors on peripheral terminals of the trigeminal nerve innervating cranial blood vessels. This action may contribute to the antimigrainous effect of sumatriptan in humans.

The formulations and methods according to the present invention may also contain additional active pharmaceutical ingredients, such as, for example, tricyclic antidepressants (e.g., amitriptyline, amoxapine, clomipramine, desipramine; doxepin; imipramine; moclobemide, nortriptyline; protriptyline; trimipramine); and other drugs including, but not limited to aspirin, beta blockers, dihydroergotamine, flunarizine, indoramine, methysergide, non-steroidal anti-inflammatories, oxetorone, pizotifene, sodium valproate, venlafaxine, and verapamil.

The formulations and methods of the invention achieve improved onset of a pharmaceutical effect of selective 5-hydroxytryptamine receptor subtype agonists by delivery through the oral mucosa of a mammal in need of treatment. In one embodiment, the 5-hydroxytryptamine receptor subtype agonist is provided in a unit dose volume of about 50 to 600 mcL, preferably about 120 to 360 mcL, and most preferably about 240 mcL.

Without being limited to any particular theory, the highly rapid onset of systemic blood concentration for formulations and methods in accordance with the invention is believed to be due at least in part to the utilization of unit dose volumes according to preferred embodiments of the invention. A unit dose volume of about 300 mcL or lower is believed to result in better absorption of the drug by the oral mucosa and less ingestion of the formulation into the GI system.

Administration of a unit dose volume of less than about 300 mcL also results in a more efficient absorption of the drug by the oral mucosa and, therefore, a lower dose of the active ingredient is needed to achieve the beneficial effects of the drug. In one embodiment, about 5 to 40 mg of a 5-hydroxytryptamine receptor subtype agonist is provided in the unit dose volume, more preferably about 10 mg to 30 mg, and most preferably about 20 mg. In one embodiment, the unit dose volume is delivered in more than one actuation of a spray pump. For example, a 20 mg dose can be delivered in two actuations of a spray pump at a unit dose volume of about 120 mcL per actuation.

Formulations and methods in accordance with preferred embodiments of the invention attain a peak blood level concentration of greater than about 5 to 10 ng/ml of the selective 5-hydroxytryptamine receptor subtype agonist within about 3 to 15 minutes post dosing, more preferably within about 5 to 12 minutes post dosing, and most preferably within about 6 minutes post dosing. In another embodiment, a second peak blood level concentration of greater than about 5-10 ng/ml of the selective 5-hydroxytryptamine receptor subtype agonist is attained within about 60 to 120 minutes post dosing, more preferably within about 90 minutes post dosing. In a preferred embodiment, a first peak blood level concentration of greater than about 10 ng/ml is achieved within about 6 minutes post dosing, and a second peak blood level concentration of greater than about 5 ng/ml is achieved within about 90 minutes post dosing.

In yet another embodiment, an area under the curve (AUC) of greater than about 0.1 to 2.0 [(ng/ml)*h] is achieved within about 3 to 12 minutes, more preferably about 4 to 10 minutes, and most preferably about 6 minutes post dosing with a selective 5-hydroxytryptamine receptor subtype agonist. In a preferred embodiment, an AUC of about 0.4 is achieved within about 6 minutes post dosing.

Under stability analyses, the storage stable compositions of the present invention also show remarkable maintenance of the initial concentration of active agent and reduced level of impurities. For example, preferred formulations of the invention (Formulation B, below) maintained sumatriptan content between a concentration of 9 mg/spray pump actuation and 11 mg/spray actuation (free base equivalents) over a 6 month period at 25° C. and 60% RH, while the average impurity concentration was less than 0.5% for a 6 month period when stored at 25° C./60% RH and less than 1.5% when stored at 40° C./75% RH. Both values are well below the limit (4%) of impurity levels of sumatriptan nasal spray, USP.

The size of the spray particles and shape of the spray pattern also may contribute to whether the active is absorbed into body systems other than the oral mucosa (e.g., lungs). For example, smaller sized particles are more likely to be inhaled. By “oral” herein we mean of, or pertaining to, the mouth and oral cavity, including but not limited to the oral mucosal surfaces of the tongue, cheeks, gums and/or sublingual surfaces.

In one embodiment, the percentage of the particles (droplets) of the spray formulation (e.g., after actuation of a spray pump) which have a volume of less than 10 microns is less than about 5%, more preferably less than about 3%. In another embodiment, the median diameter of the spray particles is from about 15 microns to about 100 microns, more preferably from about 20 microns to about 70 microns, and most preferably about 35 microns (Table 1).

TABLE 1
Particle size analysis of 11.23% sumatriptan
succinate formulations.

| Formulation | % < 10 μm | Dv(10) | Dv(50) | Dv(90) | Span |
|---|---|---|---|---|---|
| Non-taste | 2.39 | 19 | 36 | 83 | 1.8 |
| masked | | | | | |
| Taste masked | 3.04 | 17 | 33 | 66 | 1.5 |

The ovality is defined as the ratio of Dₘₐₓ and Dₘᵢₙ. Dₘₐₓ is defined as the largest chord, in mm, that can be drawn within the spray pattern that crosses the COMw (i.e., center of mass of the spray pattern) in base units. Dₘᵢₙ is described as the smallest chord, in mm, that can be drawn within the spray pattern that crosses the COMw in base units. COMw is defined as the center of mass of the detected spray pattern, where each pixel's intensity is taken into account. The ovality ratio of the spray pattern indicates whether the spray is symmetrical. It is believed that the more symmetrical the oval shape of the pattern of spray particles, the more likely the particles will evenly cover the oral mucosa. In accordance with a preferred embodiment of the invention, the ovality ratio of the pattern is less than about 2.0, more preferably less than about 1.5. The ovality ratio of the spray pattern for the taste masked formulation (Formulation B) was determined and found to be 1.46.

One preferred formulation is shown in Table 2:

TABLE 2
Taste masked sumatriptan succinate lingual
spray formulation (“Formulation A”).

| Ingredients | mg/g |
|---|---|
| Purified Water, USP | 873.25 |
| Monobasic Potassium Phosphate, NF | 5.98 |
| Sodium Hydroxide, NF | 0.07 |
| Sodium Benzoate, NF | 4.8 |
| Sucralose, NF | 2.4 |
| Sunett ® (acesulfame potassium) | 0.6 |
| N-C Mint Flavor, Nat. & Art. | 0.5 |
| Sumatriptan Succinate, EP | 112.4 |

An alternative formulation is shown in Table 3:

TABLE 3
Taste masked sumatriptan succinate lingual
spray formulation (“Formulation B”).

| Ingredients | mg/g |
|---|---|
| 50 mM Monobasic Potassium Phosphate Buffer | 845.1 |
| Sodium Benzoate, NF | 5.0 |
| Neotame | 0.1 |
| Magnasweet ® MM110 | 10.0 |
| Art. Honey Flavor | 10.0 |
| Sodium Chloride, USP | 17.5 |
| Sumatriptan Succinate, EP | 112.3 |

50 mM monobasic potassium phosphate is one preferred buffer. Other buffer strengths may be used (e.g., between 10 and 200 mM). In addition, other buffers may be used which allow for acidic solutions, in the pH range of 4.0-6.5. These buffers include acetate, carbonate, citrate, malate, propionate, and succinate. Alternatively, a buffer-free aqueous solution may be used. A preferred concentration of buffer in sumatriptan formulations in accordance with one embodiment of the invention is about 75 to 99%, more preferably about 85 to 90%.

In one embodiment of the invention, sumatriptan formulations do not contain sweetening, taste masking, or flavoring agents. However, sweetening, taste masking, and/or flavoring agents such as artificial honey flavor, bitter mask, glycyrrhizic acid, natural and artificial mint flavor, neotame, peppermint oil, sorbitol, Splenda® (sucralose), sucrose, or Sunett® (acesulfame K) can be added if desired.

Various flavors or flavoring agents may be included to impart a pleasant taste. A pleasant taste is particularly important when the formulation is intended for administration to children or animals. Numerous flavors that are commonly used in pharmaceuticals, foods, candies and beverages are also suitable for use in the present invention. Examples include beef, bubble gum, cherry, chicken, fish, fruit, honey, lemon, licorice, mint, orange, peppermint, spearmint, wintergreen, and other flavors.

Sucralose and Sunett® are preferably used to sweeten the product. Other sweeteners may be used, such as MagnaSweet®, neotame, ProSweet®, and saccharin. Flavors such as N-C mint, artificial honey flavor, or other suitable flavoring agents can be used in preferred embodiments of the invention. Other formulation options include sodium chloride to mask the bitterness of the active ingredient. Other alternatives to sodium chloride include sodium acetate and other sodium or potassium salts capable of reducing the perception of bitterness.

Alternatively, an exemplary non-taste masked formulation can be prepared according to the following formula (Table 4):

TABLE 4
Non-taste masked sumatriptan succinate oral
spray formulation (“Formulation C”)

| Ingredients | mg/g |
|---|---|
| 50 mM Monobasic Potassium Phosphate Buffer | 882.7 |
| Sodium Benzoate, NF | 5.0 |
| Sumatriptan Succinate, EP | 112.3 |

In one embodiment, the components of Formulation C have the same function as the components of Formulation A.

The formulations can contain an optional propellant for delivery as an aerosol spray or can be propellant-free and delivered by a metered valve spray pump. Suitable propellants including, but are not limited to, hydrocarbons (butane, propane, etc.), chlorofluorocarbons (CFC-11, CFC-12, etc.), hydrofluorocarbons (HFA-134a, HFA-227ea, etc.), ethers (dimethylether, diethylether, etc.), and perfluorocarbons.

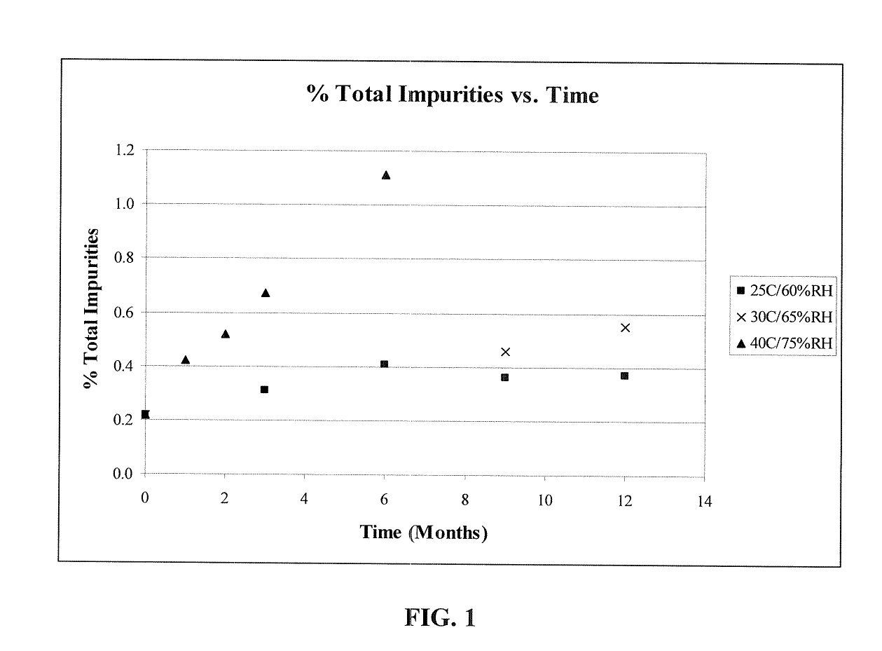

The stability of formulations in accordance with preferred embodiments of the invention are believed to be superior to the prior art and other tested formulations as discussed below and shown, for example, in Tables 5 through 7 and FIG. 1.

TABLE 5
Stability data for non-taste masked 11.23%
w/w sumatriptan succinate formulation

| | | | | Formulation | Sodium | |
|---|---|---|---|---|---|---|
| Stability | Spray | % Label | SC/SW | Content | Benzoate | Total |
| Condition | Content | Claim | ratio ¹ | (% w/w) | (% w/w) | Impurities |
| Initial | 13.56 ² | 96.90 | 0.113 ² | 11.40 ² | 0.498 | 0.15 |
| 25° C./60% RH | 13.37 ² | 95.50 | 0.113 ² | 11.31 ² | 0.502 | 0.34 |
| 1 Month | | | | | | |
| 25° C./60% RH | 9.46 ³ | 94.60 | 0.081 ³ | 11.37 ² | 0.504 | 0.30 |
| 2 Months | | | | | | |
| 25° C./60% RH | 9.30 ³ | 93.00 | 0.079 ³ | 11.17 ² | 0.505 | 0.39 |
| 3 Months | | | | | | |
| 25° C./60% RH | 9.30 ³ | 93.00 | 0.078 ³ | 7.99 ³ | 0.479 | 0.47 |
| 6 Months | | | | | | |
| 30° C./65% RH | 9.46 ³ | 94.60 | 0.080 ³ | 11.13 ² | 0.504 | 0.38 |
| 2 Months | | | | | | |
| 30° C./65% RH | 9.31 ³ | 93.10 | 0.079 ³ | 11.20 ² | 0.502 | 0.52 |
| 3 Months | | | | | | |
| 30° C./65% RH | ND | ND | ND | 7.93 ³ | 0.480 | 0.65 |
| 6 Months | | | | | | |
| 40° C./75% RH | 13.21 ² | 94.30 | 0.112 ² | 11.29 ² | 0.496 | 0.20 |
| 2 Weeks | | | | | | |
| 40° C./75% RH | 13.18 ² | 94.10 | 0.110 ² | 11.17 | 0.498 | 0.46 |
| 1 Month | | | | | | |
| 40° C./75% RH | 8.82 ³ | 88.20 | 0.078 ³ | 11.10 | 0.497 | 0.83 |
| 3 Months | | | | | | |
| 40° C./75% RH | ND | ND | ND | 7.80 ³ | 0.463 | 1.33 |
| 6 Months | | | | | | |

¹ Spray content divided by spray weight.
² Values reported and based upon succinate value.
³ Values reported and based upon free base equivalent value.
ND—not determined

TABLE 6
Stability data for taste masked 11.23% w/w sumatriptan succinate
formulation (values based on free base equivalent value).

| | | | | Formulation | Sodium | Total |
|---|---|---|---|---|---|---|
| Stability | Spray | % Label | SC/SW | Content | Benzoate | Impurities |
| Condition | Content (mg) | Claim | ratio ¹ | (% w/w) | (% w/w) | (%) |
| Initial | 9.34 | 93.4 | 0.080 | 7.83 | 0.50 | 0.12 |
| 25° C./60% RH | 9.53 | 95.3 | 0.079 | 7.96 | 0.51 | 0.17 |
| 2 Months | | | | | | |
| 25° C./60% RH | 9.51 | 95.1 | 0.079 | 7.95 | 0.50 | 0.24 |
| 3 Months | | | | | | |
| 25° C./60% RH | 9.73 | 97.3 | 0.080 | 7.99 | 0.49 | 0.24 |
| 6 Months | | | | | | |
| 25° C./60% RH | 9.20 | 92.0 | 0.079 | 7.98 | 0.50 | 0.13 |
| 9 Months | | | | | | |
| 25° C./60% RH | 9.32 | 93.2 | 0.079 | 7.91 | 0.51 | 0.37 |
| 12 Months | | | | | | |
| 30° C./65% RH | 9.64 | 96.4 | 0.079 | 8.03 | 0.50 | 0.48 |
| 9 Months | | | | | | |
| 30° C./65% RH | 9.07 | 90.7 | 0.079 | 7.92 | 0.52 | 0.58 |
| 12 Months | | | | | | |
| 40° C./75% RH | 9.24 | 92.4 | 0.078 | 7.75 | 0.48 | 0.29 |
| 1 Month | | | | | | |
| 40° C./75% RH | 9.22 | 92.2 | 0.079 | 7.88 | 0.50 | 0.39 |
| 2 Months | | | | | | |
| 40° C./75% RH | 9.35 | 93.5 | 0.078 | 7.91 | 0.50 | 0.56 |
| 3 Months | | | | | | |
| 40° C./75% RH | 9.39 | 93.9 | 0.076 | 7.82 | 0.46 | 0.85 |
| 6 Months | | | | | | |

¹ Spray content divided by spray weight.
ND—not determined

TABLE 7
Stability data for taste masked 11.23% w/w sumatriptan succinate
formulation (values based on free base equivalent value).

| | | | | Formulation | Sodium | Total |
|---|---|---|---|---|---|---|
| Stability | Spray | % Label | SC/SW | Content | Benzoate | Impurities |
| Condition | Content (mg) | Claim | ratio ¹ | (% w/w) | (% w/w) | (%) |
| Initial | 9.69 | 96.9 | 0.080 | 8.01 | 0.51 | 0.22 |
| 25° C./60% RH | 9.78 | 97.8 | 0.079 | 7.92 | 0.48 | 0.31 |
| 3 Months | | | | | | |
| 25° C./60% RH | 9.62 | 96.2 | 0.079 | 7.93 | 0.51 | 0.41 |
| 6 Months | | | | | | |
| 25° C./60% RH | 9.78 | 97.8 | 0.079 | 8.01 | 0.51 | 0.36 |
| 9 Months | | | | | | |
| 25° C./60% RH | 9.53 | 95.3 | 0.078 | 7.69 | 0.54 | 0.37 |
| 12 Months | | | | | | |
| 30° C./65% RH | 9.53 | 95.3 | 0.079 | 7.96 | 0.50 | 0.46 |
| 9 Months | | | | | | |
| 30° C./65% RH | 9.35 | 93.5 | 0.078 | 7.54 | 0.54 | 0.55 |
| 12 Months | | | | | | |
| 40° C./75% RH | 9.52 | 95.2 | 0.078 | 7.84 | 0.51 | 0.42 |
| 1 Month | | | | | | |
| 40° C./75% RH | 9.79 | 97.9 | 0.079 | 7.92 | 0.49 | 0.52 |
| 2 Months | | | | | | |
| 40° C./75% RH | 9.71 | 97.1 | 0.077 | 7.87 | 0.47 | 0.67 |
| 3 Months | | | | | | |
| 40° C./75% RH | 9.60 | 96.0 | 0.078 | 7.84 | 0.52 | 1.11 |
| 6 Months | | | | | | |

¹ Spray content divided by spray weight.
ND—not determined

As used herein, “storage stable” means liquid pharmaceutical formulations which include a selective 5-hydroxytryptamine receptor subtype agonist as an active ingredient and in which the concentration of the active ingredient is substantially maintained during storage stability testing, and degradation products and/or impurities which are typically observed in storage stability testing of such formulations are absent or significantly reduced during storage stability testing. In one embodiment, storage stability is determined at a temperature range from about 5° C. to about 80° C., more preferably from about 20° C. to about 70° C., and most preferably from about 25° C. to about 60° C. In another embodiment, storage stability is determined at a relative humidity (“RH”) range from about 30% RH to about 90% RH, more preferably from about 50% RH to about 65% RH, and most preferably from about 65% RH to about 75% RH. Preferred time intervals for measuring storage stability range from about 1 week to 3 years, more preferably from about 2 weeks to about 2 years, and most preferably at intervals of 2 weeks, 1 month, 2 months, 3 months, 6 months, 12 months, 18 months, and 24 months.

FIG. 1 shows the percent of total impurities in Formulation B under three sets of conditions (25° C./60% RH, 30° C./65% RH, and 40° C./75%RH) from 0 to 12 months. The percent impurities under all three conditions remained below 1% for six months and below 1% for the twelve months under the 25° C./60% RH and 30° C./65% RH conditions.

In accordance with one embodiment of the invention, an antimicrobial component or agent is included to ensure safe storage without the proliferation of pathogenic molds, yeasts, or bacteria. The preferred compositions use sodium benzoate as a preservative although other preservatives may be used, or preservative-free formulations can be provided in accordance with the invention. Preferably, sodium benzoate, NF is included at a concentration of about 0.01% to about 2.0%, more preferably from about 0.02% to about 1.0%, and most preferably at 0.5%. The stability of the preservative was also monitored throughout the stability study and found to remain intact (Tables 5 through 7).

In another embodiment of the invention, various antimicrobials which are suitable for use in foods and other ingestible substances are known in the art and can be used in the present invention. Examples include benzalkonium chloride (as well as other quaternary ammonium compounds), the parabens (e.g., butylparaben, methylparaben, and propylparaben), propyl-p-hydroxybenzoates, sodium benzoate, and sorbic acid including salts thereof.

The formulations of the present invention can be prepared by various methods. A preferred method of manufacturing is set forth in Table 8:

TABLE 8
Exemplary method of manufacture

| | |
|---|---|
| Step 1 | Dissolve 2.722 g of monobasic potassium phosphate, |
| | NF in purified water, USP in a 100 mL volumetric |
| | flask, to make a 100 mL of monobasic potassium |
| | phosphate solution. |
| Step 2 | Dissolve 0.08 g of sodium hydroxide, NF in purified |
| | water, USP, in a 10 mL volumetric flask to make a |
| | 10 mL of sodium hydroxide solution. |
| Step 3 | Thoroughly mix together 50 mL of the monobasic |
| | potassium phosphate solution with 2.0 mL of the |
| | sodium hydroxide solution. Dilute to 200 mL with |
| | purified water, USP to make 50 mM monobasic |
| | potassium phosphate buffer. |
| Step 4 | For large volumes, the appropriate amount of |
| | monobasic potassium phosphate, NF can be added |
| | directly to an appropriate amount of purified |
| | water, USP. The appropriate amount of sodium |
| | hydroxide, NF can then be added to the monobasic |
| | potassium phosphate solution. |

The following procedure (Table 9), among others, can be used to make preferred taste masked formulations.

TABLE 9
Exemplary procedure to make taste-masked formulations

| | |
|---|---|
| Step 1 | While stirring, add sodium benzoate, NF to the |
| | 50 mM monobasic potassium phosphate buffer. |
| | Continue to stir until the solution is clear and |
| | particle free. |
| Step 2 | While stirring the above solution, add the |
| | sweeteners one at a time. Continue stirring until |
| | the sweeteners are dissolved or incorporated and |
| | the solution is well mixed. |
| Step 3 | When the solution above is clear, add the flavor |
| | while stirring. Continue stirring until the |
| | solution is well mixed. |
| Step 4 | When the solution above is clear, and if |
| | appropriate, add the sodium chloride, USP while |
| | stirring. Continue to stir until the sodium |
| | chloride is dissolved and the solution is clear |
| | and particle free. |
| Step 5 | When the above solution is clear and particle |
| | free, add sumatriptan succinate, EP while |
| | stirring. In the case of making small batches, |
| | the sumatriptan succinate, EP may need to be added |
| | in increments. In this case, allow the solution to |
| | clear before making subsequent additions. |
| Step 6 | Stir until solution is completely clear and in one |
| | phase. |
| Step 7 | Filter the solution through appropriately sized |
| | filter. |

The solution can then be packaged into any suitable containers. Preferred containers are pharmaceutically acceptable glass, PET, and HDPE bottles with a capacity of between 1 and 100 mL. To ensure long-term photostability amber glass can be utilized. Additionally, if PET or HDPE is chosen, the bottle may be opaque to ensure long-term photostability.

The formulations are preferably dispensed using a metered pump device capable of delivering between 10 and 500 mcL. Pumps commonly used for dispensing nasal sprays are suitable for use with these formulations. In one embodiment, the pump and actuator may be modified such that the spray is dispensed horizontally to the bottle. This will allow easy dispensing to the mouth of the patient. The actuator may include an extension, if desired, to facilitate delivery to the buccal area of humans or cheek pouches of animal.

The present invention also provides methods of treating various conditions in a subject (e.g., treatment of acute migraine attacks and cluster headaches). The methods of treatment include administering to a subject in need of treatment a storage stable pharmaceutical composition according to the invention. In one embodiment, the subject is a human; in another embodiment the subject is a non-human mammal selected from the group of dogs, cats, horses, cattle, sheep, and swine. The storage stable pharmaceutical composition can be administered to a patient in any suitable dosage range, including a dosage range of, for example, 5 mg to about 40 mg per dose, and more preferably 10 to 30 mg per dose. The storage-stable pharmaceutical composition can also be delivered in any suitable unit dose volume, preferably about 50 mcL to about 600 mcL, more preferably from about 120 to 360 mcL, and most preferably about 240 mcL.

It is to be understood that application of the teachings of the present invention to a specific problem or environment will be within the capability of one having ordinary skill in the art in light of the teachings contained herein. The present invention is more fully illustrated by the following non-limiting examples.

### EXAMPLE 1

Pharmacokinetic parameters for oral mucosal spray delivery and oral tablet administration of sumatriptan formulations were measured and evaluated in three groups of ten healthy male volunteers. Each member of Group I was administered a 50 mg sumatriptan tablet. Each member of Group II received a lingual spray dose of 20 mg of Sumatriptan Formulation B. Each member of Group III received a lingual spray dose of 30 mg of Sumatriptan Formulation B. The 20 mg lingual spray dose was provided in a spray volume of 240 mcL while the 30 mg lingual spray dose was provided in a spray volume of 360 mcL.

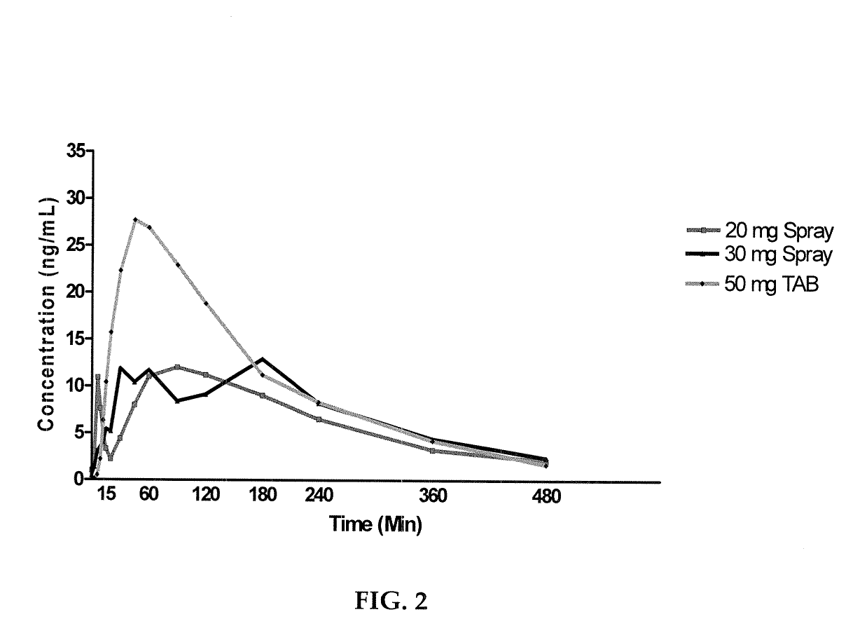

As shown in FIG. 2, administration of the 20 mg lingual formulation resulted in a first peak blood concentration of about 11 ng/ml at about six minutes post dosing and a second peak blood concentration of about 12 ng/ml at about 90 minutes post dosing. In contrast, the 50 mg tablet dose each resulted in a single peak blood concentration of about 27 ng/ml at about 1 hour post dosing.

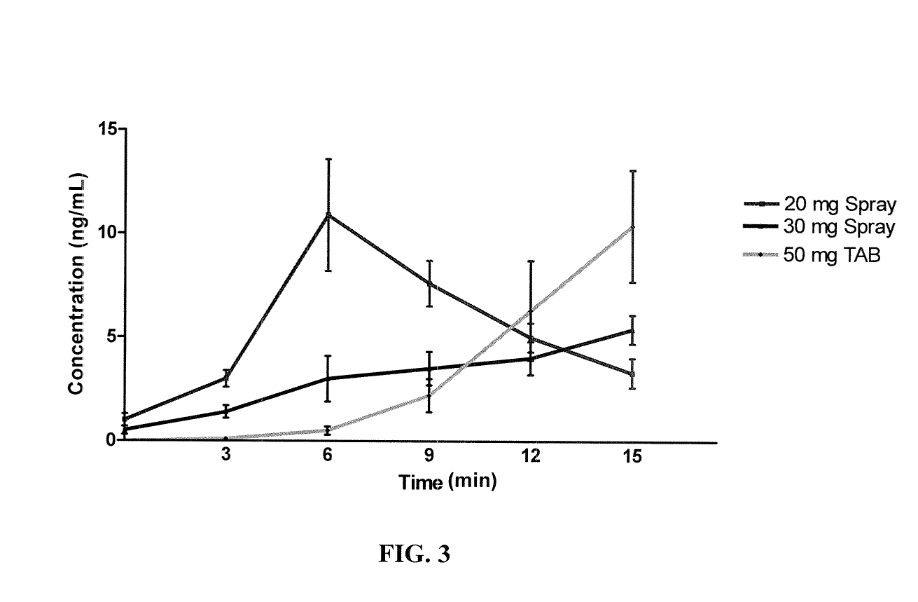

FIG. 3 illustrates the exemplary fast onset properties of the sumatriptan formulations in accordance with the invention. As shown in FIG. 3, administration of the 20 mg lingual spray formulation resulted in a blood concentration of about 12 ng/ml at about six minutes post dosing. In contrast, the 30 mg lingual spray formulation resulted in a blood concentration of about 3 ng/ml at about six minutes post dosing. Administration of the 50 mg tablet resulted in a negligible peak blood concentration at about six minutes post dosing and did not reach 10 ng/ml until fifteen minutes post dosing. Thus, formulations in accordance with preferred embodiments of the invention result in rapid onset of drug.

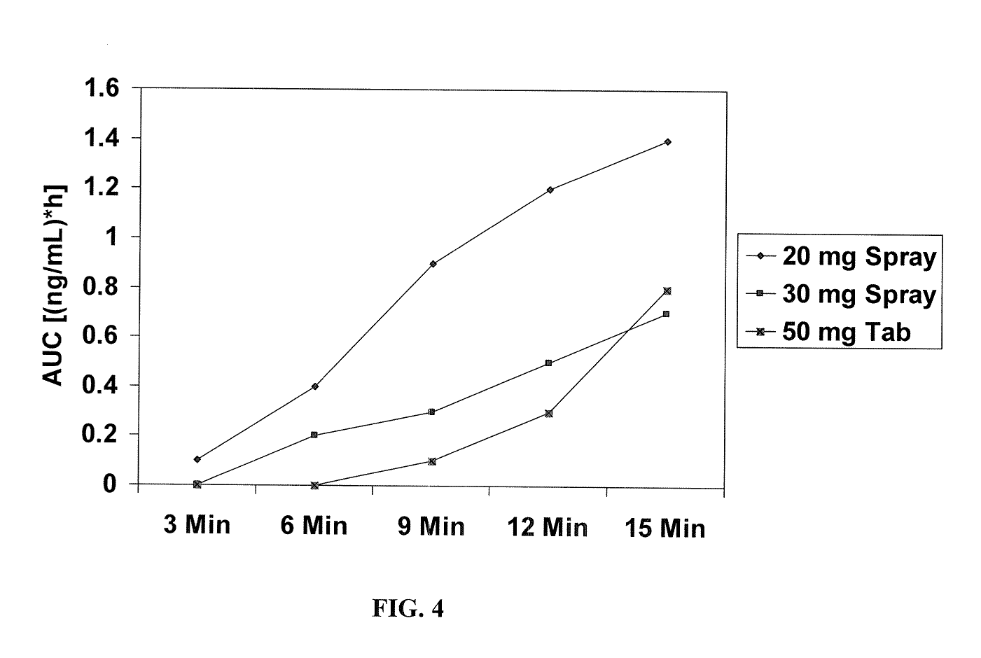

FIG. 4 shows the total amount of drug absorbed by measuring the area under the curve or AUC. The AUC for the 20 mg lingual spray formulation (0.4 [(ng/ml)*h]) is twice as great as the AUC for the 30 mg lingual spray formulation (0.2 [(ng/ml)*h] at six minutes post dosing. The AUC for the 50 mg tablet formulation is zero at six minutes post doing. At nine minutes post dosing, the AUC for the 20 mg lingual spray formulation is about 4 times the AUC for the 30 mg lingual spray formulation and about eight times the AUC for the 50 mg formulation.

These results (FIGS. 2-4) demonstrate a significantly faster rate of drug absorption for the oral spray formulations, particularly the preferred 20 mg, 240 mcL dose, compared to the tablet formulation, and up to a 50% increase in relative bioavailability of sumatriptan. The first peak plasma concentration of sumatriptan was approximately 70% faster with the 20 mg lingual spray formulation than with Imitrex nasal spray.

### EXAMPLE 2

A multiple treatment, randomized, 5-way crossover comparative study evaluating the rate and extent of pain relief after administration of sumatriptan lingual spray formulations and sumatriptan tablets was conducted. Thirty-seven migraine sufferers completed at least two treatment arms. 50 mg and 100 mg sumatriptan tablets and 20, 30, and 40 mg lingual spray formulations were used in the study.

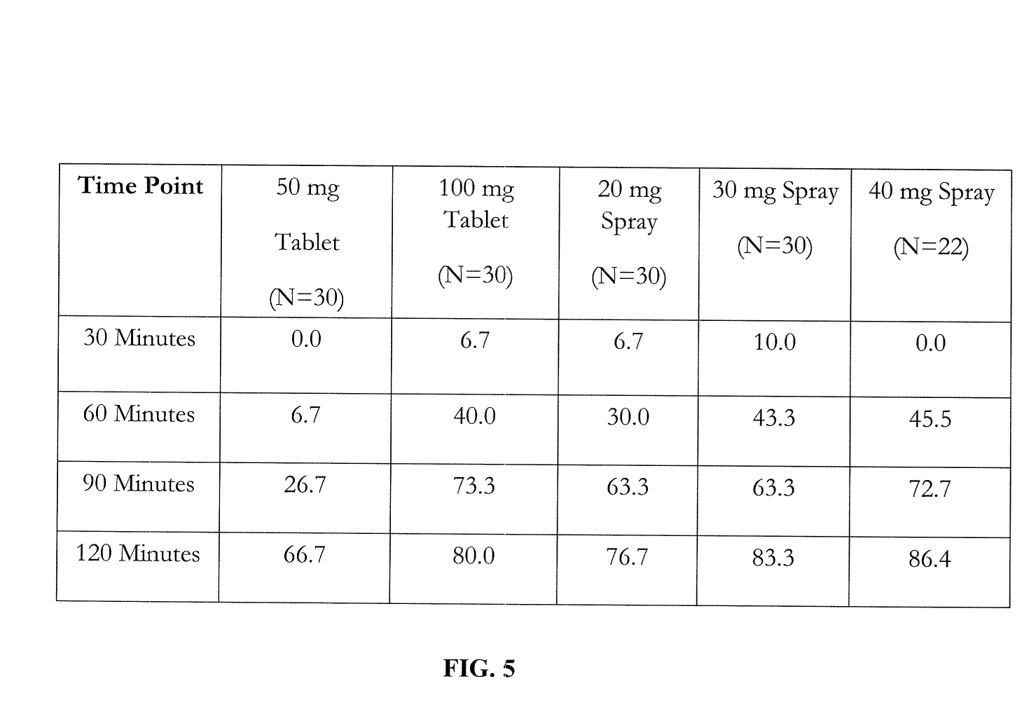

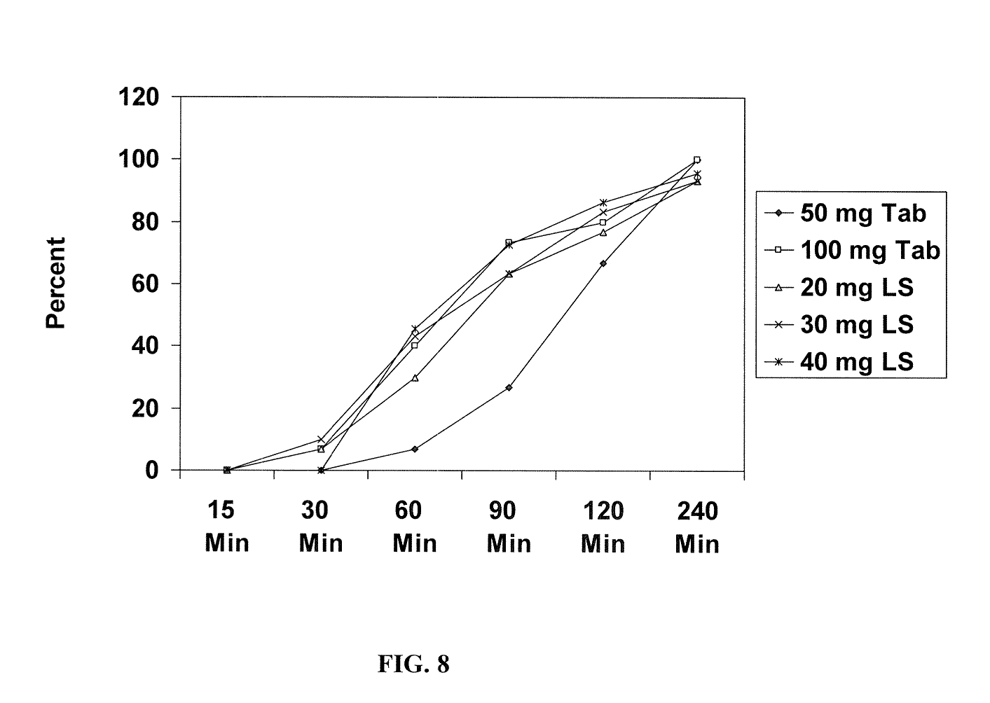

FIG. 5 shows the percentage of subjects with a headache response which is defined as at least a two point reduction in headache severity rating from 30 to 120 minutes after treatment. Subjects completed four treatment arms. Thirty minutes following treatment, 6.7% of the subjects receiving the 100 mg tablet and the 20 mg lingual spray had a headache response while 10% of the subjects receiving the 30 mg lingual spray had a headache response. After sixty minutes, the headache response ranged from 6.7% for the 50 mg tablet to 45.5% for the 40 mg spray. After ninety minutes, 63.3% of the subjects reported a response for the 20 and 30 mg spray formulations. FIG. 8 shows the rate of response to treatment for the various doses. All of the doses provided a significantly faster rate of response than the 50 mg tablet. The 20 mg spray dose combines a fast response with a low dose.

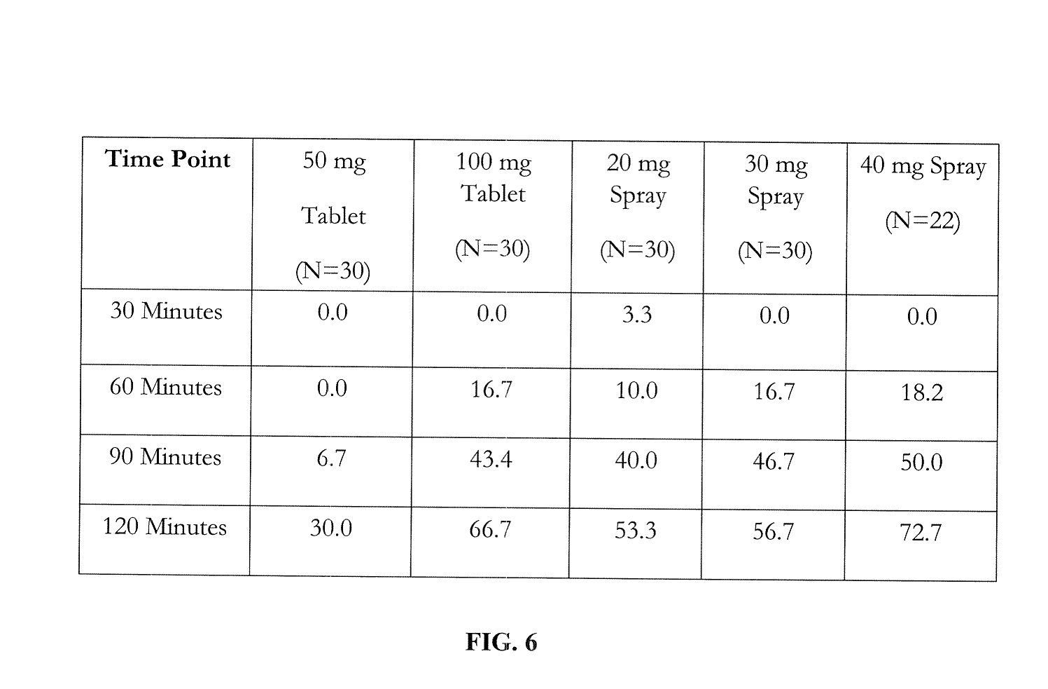

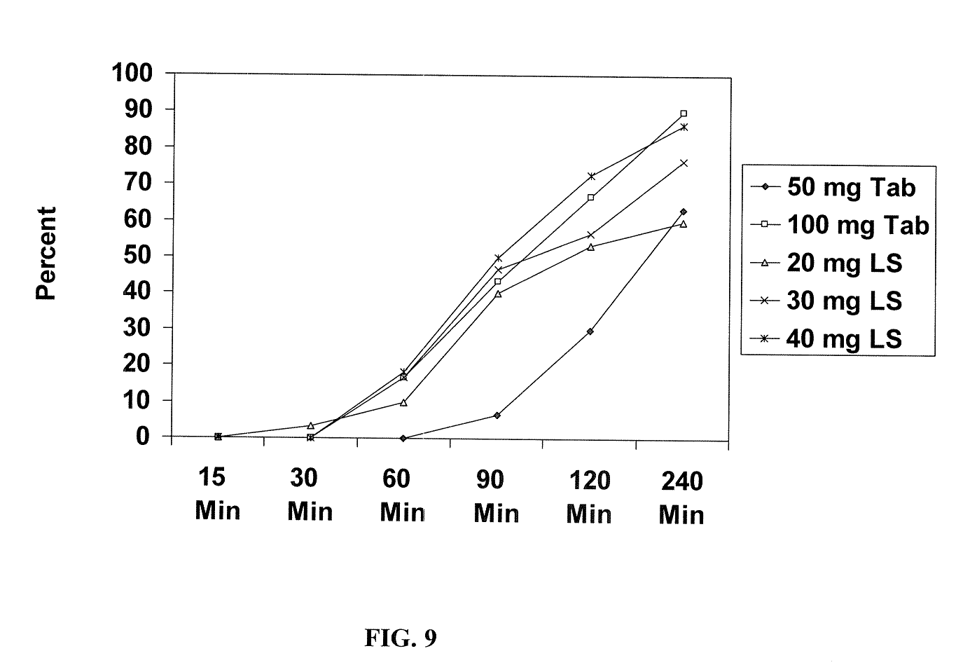

FIG. 6 shows the percentage of subjects with complete pain relief. Thirty minutes after treatment, only the 20 mg spray resulted in complete pain relief (3.3%). After sixty minutes, the 100 mg tablet and 30 mg spray resulted in 16.7% of subjects reporting complete pain relief. Within the first sixty minutes, no subjects reported complete pain relief after receiving the 50 mg tablet. FIG. 9 shows the rate of complete pain relief response. The 20 mg spray provided a significantly faster rate of complete pain relief than the 50 mg tablet. As with the rate of response, the 20 mg spray combines a fast onset of complete pain relief with a low dose.

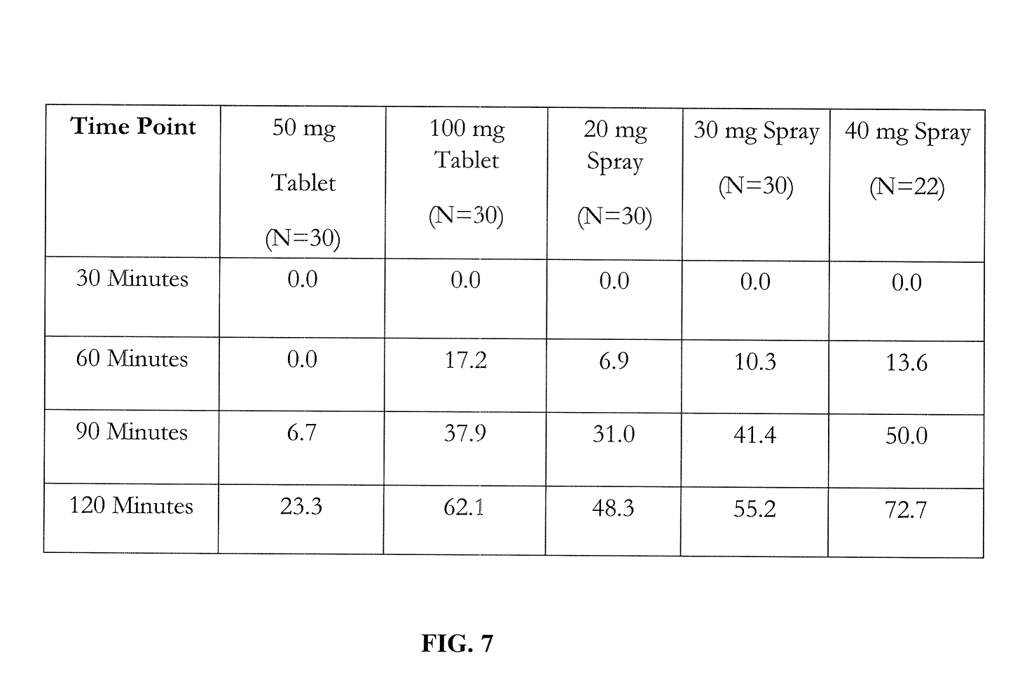

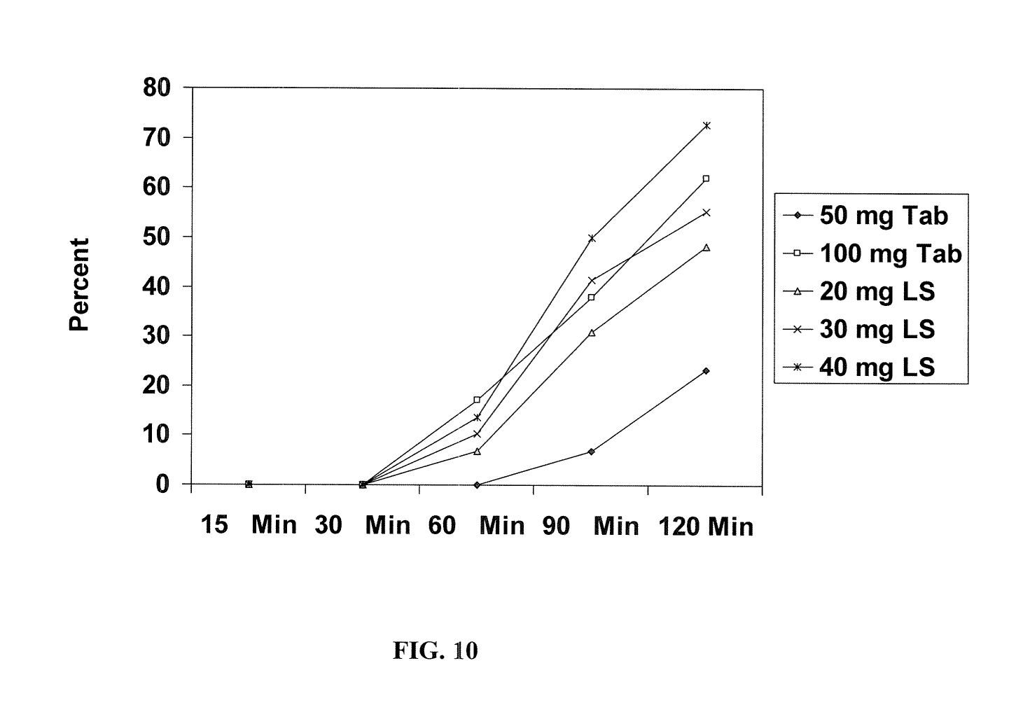

FIG. 7 shows the percentage of subjects reporting no disability (defined as complete pain relief with no associated symptoms) after receiving the indicated formulations. After sixty minutes, none of the subjects reported absence of disability after receiving the 50 mg tablet while 6.9% of the subjects receiving the 20 mg spray reported absence of disability. Thus, the 20 mg spray provides faster onset of complete pain relief with no symptoms than the 50 mg tablet at more than half the dose. Furthermore, as shown in FIG. 10, the rate of no disability response is significantly faster in all doses compared to the 50 mg tablet.

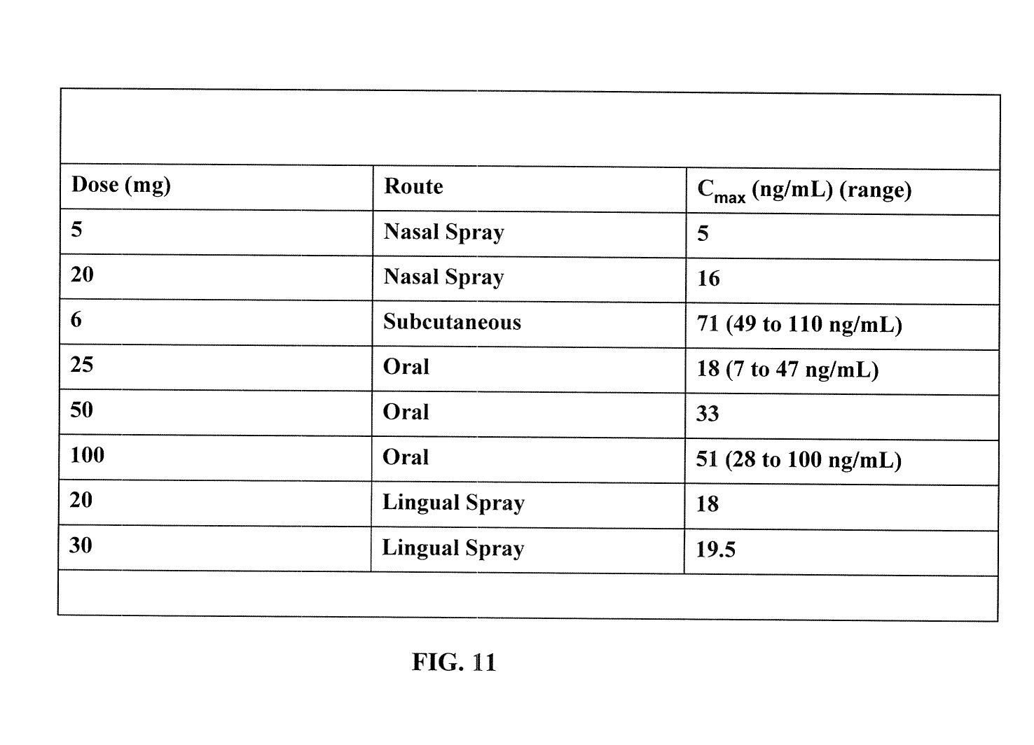

FIG. 11 shows the Cmax (i.e., the maximum concentration of a drug in the body after dosing) for various dose formulations including the 50 and 100 mg tablet and the 20 and 30 mg lingual spray formulations. The lingual spray doses achieve a significantly lower Cmax than the tablet formulations, yet, as shown above, provide fast onset of pain relief.

The above description and examples are only illustrative of preferred embodiments which achieve one or more objects, features, and advantages of the present invention, and it is not intended that the present invention be limited thereto. Any modifications of the present invention which come within the spirit and scope of the following claims is considered part of the present invention.

What is claimed as new and desired to be protected by Letters Patent of the United States is:

1. An oral spray composition, comprising a selective 5-hydroxytryptamine receptor subtype agonist, wherein when a unit dose volume of about 50 to 600 mcL of the oral spray composition is sprayed on the oral mucosal surface of a human or non-human animal, a first peak blood concentration of the selective 5-hydroxytryptamine receptor subtype agonist is greater than about 5 to 10 ng/ml within about 3 to 15 minutes post-dosing.

2. The composition of claim 1, wherein when the unit dose volume is sprayed on the oral mucosal surface of a human or non-human animal, a second peak blood concentration of the selective 5-hydroxytryptamine receptor subtype agonist is greater than about 5 to 10 ng/ml of the selective 5-hydroxytryptamine receptor subtype agonist within about 60 to 120 minutes post dosing.

3. The composition of claim 1, wherein the first peak blood concentration of the selective 5-hydroxytryptamine receptor subtype agonist is greater than about 5 to 10 ng/ml within about 5 to 12 minutes post dosing.

4. The composition of claim 1, wherein the first peak blood level concentration of the selective 5-hydroxytryptamine receptor subtype agonist is greater than about 10 ng/ml within about 6 minutes post dosing, and a second peak blood level concentration of greater than about 10 ng/ml is achieved within about 90 minutes post dosing.

5. The composition of claim 3, wherein the first peak blood concentration of the selective 5-hydroxytryptamine receptor subtype agonist is greater than about 5 to 10 ng/ml within about 6 minutes post dosing.

6. The composition of claim 1, wherein the unit dose volume is about 120 to 360 mcL.

7. The composition of claim 6, wherein the unit dose volume is about 240 mcL.

8. The composition of claim 1, wherein the unit dose volume comprises about 5 to about 40 mg of the selective 5-hydroxytryptamine receptor subtype agonist.

9. The composition of claim 8, wherein the unit dose volume comprises about 10 to about 30 mg of the selective 5-hydroxytryptamine receptor subtype agonist.

10. The composition of claim 8, wherein the unit dose volume comprises about 20 mg of the selective 5-hydroxytryptamine receptor subtype agonist.

11. The composition of claim 1, wherein the selective 5-hydroxytryptamine receptor subtype agonist is sumatriptan.

12. The composition of claim 11, wherein the concentration of sumatriptan is about 5 to 20% w/w.

13. The composition of claim 12, wherein the concentration of sumatriptan is about 7 to 15% w/w.

14. The composition of claim 13, wherein the concentration of sumatriptan is about 11% w/w.

15. The composition of claim 11, wherein said composition further comprises a buffer.

16. The composition of claim 15, wherein said buffer is selected from the group consisting of acetate, carbonate, citrate, malate, propionate, phosphates, succinate, and salts thereof.

17. The composition of claim 11, wherein said composition has a pH from about 4 to 6.5.

18. The composition of claim 11, wherein said composition is storage stable.

19. The composition of claim 11, wherein the spray volume comprises particles, less than 10% of which have a volume of less than about 10 microns.

20. The composition of claim 19, wherein the median diameter of the particles is about 15 to 100 microns.

21. The composition of claim 20, wherein the median diameter of the particles is from about 20 to about 70 microns.

22. The composition of claim 21, wherein the median diameter of the particles is about 35 microns.

23. The composition of claim 11, comprising about 10 to 15% w/w of sumatriptan succinate, about 85 to 90% of 50 mM monobasic potassium phosphate buffer, and about 0.5% w/w sodium benzoate.

24. An oral spray composition comprising a selective 5-hydroxytryptamine receptor subtype agonist selected from the group consisting of sumatriptan, almotriptan, eletriptan, frovatriptan, naratriptan, rizatriptan and zolmitriptan, about 75 to 99% of 50 mM monobasic potassium phosphate buffer, and about 0.5% w/w sodium benzoate.

25. An apparatus comprising a spray pump and a bottle, wherein the bottle contains a composition comprising about 10 to 15% w/w of sumatriptan succinate, about 85 to 90% of 50 mM monobasic potassium phosphate buffer, and about 0.5% w/w sodium benzoate, said spray pump being capable of delivering a unit dose volume of about 50 to 600 mcL of the composition.

26. The apparatus of claim 25, wherein the unit dose volume is about 120 mcL.

27. The apparatus of claim 25, wherein the spray pump is actuated more than once.

28. The apparatus of claim 25, wherein when the unit dose volume is sprayed on the oral mucosal surface of a human or non-human animal, a first peak blood concentration of the selective 5-hydroxytryptamine receptor subtype agonist is greater than about 5 to 10 ng/ml within about 3 to 15 minutes post-dosing.

29. The apparatus of claim 28, wherein when the unit dose volume is sprayed on the oral mucosal surface of a human or non-human animal, a second peak blood concentration of the selective 5-hydroxytryptamine receptor subtype agonist is greater than about 5 to 10 ng/ml of the selective 5-hydroxytryptamine receptor subtype agonist within about 60 to 120 minutes post dosing.

30. A method of treating a condition in a human or non-human animal, comprising spraying a unit dose volume of about 50 to 600 mcL of an oral spray composition comprising a selective 5-hydroxytryptamine receptor subtype agonist, wherein the selective 5-hydroxytryptamine receptor subtype agonist is absorbed through the oral mucosa to provide a peak blood concentration of the selective 5-hydroxytryptamine receptor subtype agonist greater than about 5 to 10 ng/ml within about 3 to 15 minutes post-dosing to alleviate said condition.

31. The method of claim 30, wherein when the unit dose volume is sprayed on the oral mucosal surface of a human or non-human animal, a second peak blood concentration of the selective 5-hydroxytryptamine receptor subtype agonist is greater than about 5 to 10 ng/ml of the selective 5-hydroxytryptamine receptor subtype agonist within about 60 to 120 minutes post dosing.

32. The method of claim 30, wherein the first peak blood concentration of the selective 5-hydroxytryptamine receptor subtype agonist is greater than about 5 to 10 ng/ml within about 5 to 12 minutes post dosing.

33. The method of claim 32, wherein the first peak blood concentration of the selective 5-hydroxytryptamine receptor subtype agonist is greater than about 5 to 10 ng/ml within about 6 minutes post dosing.

34. The method of claim 30, wherein the area under the curve (AUC) for the selective 5-hydroxytryptamine receptor subtype agonist is greater than about 0.4 [(ng/ml)*h] after about six minutes post-dosing.

35. The method of claim 34, wherein the area under the curve (AUC) for the selective 5-hydroxytryptamine receptor subtype agonist is greater than about 0.8 [(ng/ml)*h] after about nine minutes post-dosing.

36. The method of claim 35, wherein the area under the curve (AUC) for the selective 5-hydroxytryptamine receptor subtype agonist is greater than about 1.2 [(ng/ml)*h] after about twelve minutes post-dosing.

37. The method of claim 36, wherein the area under the curve (AUC) for the selective 5-hydroxytryptamine receptor subtype agonist is greater than about 1.4 [(ng/ml)*h] after about fifteen minutes post-dosing.

38. The method of claim 30, wherein the unit dose volume is about 50 to 250 mcL.

39. The method of claim 38, wherein the unit dose volume is about 240 mcL.

40. The method of claim 30, wherein the unit dose volume comprises about 5 to about 40 mg of the selective 5-hydroxytryptamine receptor subtype agonist.

41. The method of claim 40, wherein the unit dose volume comprises about 10 to about 30 mg of the selective 5-hydroxytryptamine receptor subtype agonist.

42. The method of claim 41, wherein the unit dose volume comprises about 20 mg of the selective 5-hydroxytryptamine receptor subtype agonist.

43. The method of claim 38, wherein the selective 5-hydroxytryptamine receptor subtype agonist is sumatriptan.

44. The method of claim 43, wherein the concentration of sumatriptan is about 5 to 20% w/w.

45. The method of claim 44, wherein the concentration of sumatriptan is about 7 to 15% w/w.

46. The method of claim 45, wherein the concentration of sumatriptan is about 11% w/w.

47. The method of claim 43, wherein the spray volume comprises particles, less than 10% of which have a volume of less than about 10 microns.

48. The method of claim 47, wherein the median diameter of the spray particles is about 15 to 100 microns.

49. The method of claim 48, wherein the median diameter of spray particles is from about 20 to about 70 microns.

50. The method of claim 49, wherein the median diameter of the spray particles is about 35 microns.

51. The method of claim 30, wherein the oral spray composition comprising about 10 to 15% w/w of sumatriptan succinate, about 85 to 90% of 50 mM monobasic potassium phosphate buffer, and about 0.5% w/w sodium benzoate.

52. The method of claim 43, wherein the sumatriptan is administered in a dose from about 5 mg to about 40 mg.

53. The method of claim 52, wherein the sumatriptan is administered in a dose from about 10 to 30 mg.

54. The method of claim 30, wherein the condition is selected from the group consisting of prophylactic therapy of migraine and the management of hemiplegic or basilar migraine, and the composition comprises about 11% w/w of sumatriptan succinate, about 90% of 50 mM monobasic potassium phosphate buffer, and about 0.5% w/w sodium benzoate.

55. A method of treating a condition in a human or non-human animal, comprising spraying a unit dose volume of about 50 to 600 mcL of an oral spray composition comprising about 10 to 15% w/w of sumatriptan succinate, about 85 to 90% of 50 mM monobasic potassium phosphate buffer, and about 0.5% w/w sodium benzoate.

56. The method of claim 55, wherein the sumatriptan is administered in a dose of about 20 mg.

57. The method of claim 55, wherein the unit dose volume is about 240 mcL.

58. The method of claim 55, wherein the composition comprises about 11% w/w of sumatriptan succinate.

59. A method of treating a condition in a human or non-human animal, comprising spraying a unit dose volume of about 50 to 600 mcL of an oral spray composition comprising about 1 to 15% w/w of a selective 5-hydroxytriptamine receptor subtype agonist is selected from the group consisting of sumatriptan, almotriptan, eletriptan, frovatriptan, naratriptan, rizatriptan and zolmitriptan, about 75 to 99% of 50 mM monobasic potassium phosphate buffer, and about 0.5% w/w sodium benzoate.
